(19) United States
(12) Patent Application Publication
(10) Pub. No.: US 2013/0096669 A1
(43) Pub. Date: Apr. 18, 2013

(54) PARTIALLY ANNEALED STENT

(71) Applicants: Rainer Bregulla, Balingen (DE); Randolf Von Oepen, Aptos, CA (US); Pamela A. Kramer-Brown, San Jose, CA (US); Carl P. Frick, Laramie, WY (US); Austin M. Leach, Oakland, CA (US)

(72) Inventors: Rainer Bregulla, Balingen (DE); Randolf Von Oepen, Aptos, CA (US); Pamela A. Kramer-Brown, San Jose, CA (US); Carl P. Frick, Laramie, WY (US); Austin M. Leach, Oakland, CA (US)

(73) Assignee: ABBOTT CARDIOVASCULAR SYSTEMS, INC., Santa Clara, CA (US)

(21) Appl. No.: 13/271,513

(22) Filed: Oct. 12, 2011

(51) Int. Cl. A61F 2/82; B23P 15/00; C21D 8/10; B21C 37/06

(52) U.S. Cl. 623/1.16; 72/340; 148/559

(57) ABSTRACT

A stent and method for manufacturing a stent that achieves both strength as well as ductility. In the manufacturing process, the material used to form the stent is only partially annealed to lower the grain size across the thickness of the stent. The material is partially annealed either prior to or after the cutting a stent pattern into a tube.

### BACKGROUND

1. The Field of the Invention

The present invention is generally directed to a method of manipulating the performance characteristics of a metal stent, and more particularly pertains to a heat treatment process for achieving a desired combination of strength and ductility.

2. The Relevant Technology

A focus of recent development work in the treatment of heart disease has been directed to endoprosthetic devices referred to as stents. Stents are generally tubular shaped devices that function to maintain patency of a segment of a blood vessel or other body lumen such as a coronary artery. They also are suitable for use to support and hold back a dissected arterial lining that can occlude the fluid passageway. At present, there are numerous commercial stents being marketed throughout the world. Intraluminal stents implanted via percutaneous methods have become a standard adjunct to balloon angioplasty in the treatment of atherosclerotic disease. Stents prevent acute vessel recoil and improve the long term outcome by controlling negative remodeling and supporting vessel dissections. Amongst their many properties, stents must have adequate mechanical strength, flexibility, minimal recoil, and occupy the least amount of arterial surface area possible while not having large regions of unsupported area.

One method and system developed for delivering stents to desired locations within the patient's body lumen involves crimping a stent about an expandable member, such as a balloon on the distal end of a catheter, advancing the catheter through the patient's vascular system until the stent is in the desired location within a blood vessel, and then inflating the expandable member on the catheter to expand the stent within the blood vessel. The expandable member is then deflated and the catheter withdrawn, leaving the expanded stent within the blood vessel, holding open the passageway thereof.

Stents are typically formed from biocompatible metals and alloys, such as stainless steel, nickel titanium, platinum iridium alloys, cobalt chromium alloys and tantalum. Such stents provide sufficient hoop strength to perform the scaffolding function. Furthermore, stents should have minimal wall thicknesses in order to minimize blood flow blockage. Starting stock for manufacturing stents is frequently in the form of stainless steel or cobalt-chromium alloy tubing, although the technology has began to explore other alloys and metals in search of the optimum balance of desirable characteristics and costs.

The performance characteristics of a stent are largely driven by the material properties of the stent material. Material properties such as strength and ductility are key in determining how the stent will behave under implanted conditions. As an example, a stent material with greater ductility will generally result in a stent that is capable of higher allowable deformation during expansion while a stent material with increased strength will usually result in a stent with increased radial rigidity. Other properties, such as elastic modulus and yield strength also have significant impacts on stent performance characteristics. Typically, however, strength and ductility are inversely related, and it is necessary to find a way to balance them by either changing the stent dimensions, configuration, or using a different material in its construction.

One important principle concerning the metallurgical consequences of processing the metals is that the structural properties of the material used for stents can improve with a decrease in the grain size of the substrate material. For example, it has been observed that stents cut from fully annealed 316L stainless steel tubing having less than seven grains across a strut thickness can display micro cracks in the high strain regions of the stent. Such cracks are suggestive of undesirable heavy slip band formation, with subsequent decohesion of the atoms along the slip planes. Reduction of the grain size in the substrate material will reduce the occurrence of such cracks and/or heavy slip band formation in the finished medical device.

Thus, in this case smaller grain size, leading to more grains across the strut thickness, limit the formation of slip bands. The grain size of a finished stainless steel or similar metal tube depends on numerous factors, including the length of time the material is heated above a temperature that allows significant grain growth. For a metallic tube, if the grain size is larger than desired, the tube may be swaged to introduce heavy dislocation densities, then heat treated to recrystallize the material into finer grains. Alternatively, different material forms may be taken through a drawing or other working and heat treat processes to recrystallize the tubing and smaller grains. The type and amount of working allowed depends on the material, e.g., ceramics may require a high temperature working step while metals and composites may be workable at room temperature. Grain-size strengthening occurs where there is an increase in strength of a material due to a decrease in the grain size. The outer diameter of the tube used to form the stent usually requires a machining step of some sort to smooth the surface after the swaging process, and the same may be true before the tubing can be properly drawn.

Commercially available 316L stainless steel tubing contains average grain sizes ranging from approximately 0.0025 inch (sixty four microns), ASTM grain size 5 to around 0.00088 inch (twenty two microns), ASTM grain size 8. These grain sizes result in anywhere from two to five grains across the tube thickness, and the stent subsequently manufactured from the tubing, depending on the tube and stent strut thicknesses. Part of the limitation in achieving a finer grain size in this material arises from the number of draws and anneals the tubing must go through to achieve its final size.

As indicated above, stents have been formed in the past by laser-cutting a small mesh structure from a tube of material. The tubing is typically formed to given dimensions through a drawing process that imparts a significant amount of work-hardening in the material. This involves an introduction of dislocations in the grains of the material through cold or warm working below a stress-relief temperature. In the case of large dimension reduction, the internal metallic grains become compacted and elongated. Both work hardening and grain size reduction limits dislocation mobility (the “Hall-Petch” relationship), causing an increase in material strength, but a severe loss of ductility. Therefore, internal stress caused by this process is then relieved through a heat treatment termed “full annealing” that greatly reduces the dislocation density and creates a homogeneous grain structure.

Stents have heretofore been formed of materials that have been fully annealed (and the material recrystallizes) either before or after grain growth. That is, the material is heated beyond its stress-relief temperature for a period of time sufficient to ensure recrystallization and a homogeneous grain structure. This process has been effective for the manufacture of common stent materials such as stainless steel and cobalt-chromium alloys, but may not be adequate to balance stent characteristics using newer materials such as tantalum and other refractory metal alloys. Therefore, there is a need for an improved method of manufacture of stent implants that provides a better balance of stent material strength and ductility.

### BRIEF SUMMARY

The present invention provides for a stent manufacturing process which obviates the need to alter the stent configuration or to select a different material for its manufacture in order to achieve a desired balance of strength and ductility. Moreover, such a process allows materials to be used in the manufacture of stents that have previously been found to exhibit an undesirable balance of strength and ductility. The process results in a material that is only partially recrystallized and that has an inhomogenous grain structure which has unexpectedly been found to yield a more desired balance of physical characteristics.

One method of the present invention provides for the partial annealing of the stent material. In one embodiment, the tubing is partially annealed before laser cutting a pattern in the tubing. In another embodiment, a stent pattern is laser cut into tubing after which the structure is partially annealed. As a further embodiment, manufacturing sequences may include a full annealing step as long as it is followed by further cold working and a final partial annealing step.

The method of the present invention allows stents to be manufactured from a wider assortment of materials including certain refractory metals and refractory metal alloys that have heretofore been found to be unsuitable for stent applications. Such materials include, but are not limited to, tantalum alloys, niobium alloys, and molybdenum alloys, including tantalum-niobium-tungsten alloys.

In one embodiment, a tantalum alloy may includes a tantalum content of about 77 weight % (“wt %”) to about 92 wt %, a niobium content of about 7 wt % to about 13 wt %, and a tungsten content of about 1 wt % to about 10 wt %.

These and other features and advantages of the present invention will become apparent from the following detailed description of the preferred embodiments which, taken in conjunction with the accompanying drawings, illustrate by way of example the principles of the present invention.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 is an elevational view, partially in section, of a fine grain stent embodying features of the invention, wherein the stent is mounted on an over-the-wire delivery catheter and a fine grain guide wire.

FIGS. 2 and 3 are cross-sectional views of the catheter assembly of FIG. 1.

FIG. 4 is cross-sectional view of a fine grain stent embodying features of the invention, wherein the stent is expanded within an artery, so that the stent apposes an arterial wall.

FIG. 5 is a cross-sectional view of an expanded fine grain stent embodying features of the invention, wherein the stent is implanted within an artery after withdrawal of a delivery catheter.

FIG. 6 is an elevated, perspective view of a fine grain stent embodying features of the invention, wherein the stent is in an unexpanded state.

FIG. 7 is an elevated perspective view of the fine grain stent of FIG. 6 in an expanded condition, depicting cylindrical rings connected by undulating links.

FIG. 8A is a graph of ultimate tensile strength (UTS) and yield strength (YS) for different annealing parameters for a fine grain stent; and

FIG. 8B is a graph of elongation (%) with different annealing parameters for a fine grain stent.

### DETAILED DESCRIPTION

Stents are well known in the art and can have many different types of patterns and configurations. The following description of intravascular stents include typical stent patterns made from a metallic tubing. Many stent patterns are well known in the art, and the description herein of stents and delivery systems is by way of example and is not meant to be limiting.

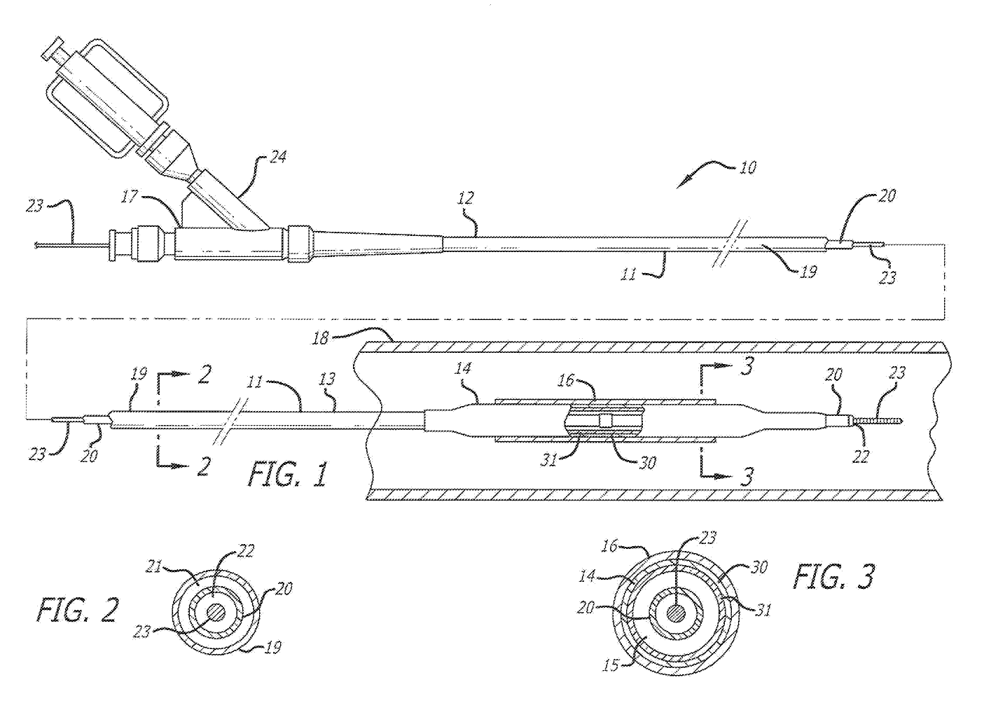

Referring to FIG. 1, a stent 16 constructed from a partially annealed material may be mounted on a catheter assembly 10, which is used to deliver the stent 16 and implant it in a body lumen 18, such as a coronary artery, peripheral artery, or other vessel or lumen within the body. The catheter assembly includes a catheter shaft 11, which has a proximal end 12 and a distal end 13. The catheter assembly is configured to advance through the patient's vascular system by advancing over a guide wire 23 by any of the well known methods utilizing an over the wire system such as the one shown in FIG. 1, or a rapid exchange (RX) catheter system (not shown). The guide wire 23 may also be constructed from a partially annealed material according to the processes of the present invention.

The proximal end of the catheter assembly 10 may be fitted with an adapter 17 that includes a guide wire port and an inflation port at a sidearm 24. The distal end of the guide wire 23 exits the catheter distal end so that the catheter advances along the guide wire. As is known in the art, a guide wire lumen 22 is configured and sized for receiving various diameter guide wires to suit a particular application. The partially annealed stent 16 is typically mounted on an expandable member (balloon) 14 positioned proximate the catheter distal end 13. The stent 16 is crimped tightly thereon, so that the stent and expandable member 14 present a low profile diameter for delivery through the patient's vasculature. The stent 16 may be used to repair a diseased or damaged arterial wall 18, a dissection or a flap that are commonly found in the coronary arteries, peripheral arteries and other vessels. The presence of arterial plaque (not shown) may be treated by an angioplasty or other repair procedure prior to stent implantation.

In a typical procedure to implant a stent 16 formed from a partially annealed material, the guide wire 23 is advanced through the patient's vascular system by well known methods so that the distal end of the guide wire is in the body lumen 18 at the designated area. Prior to implanting the stent, the cardiologist may wish to perform an angioplasty procedure or other procedure (e.g., atherectomy) in order to open the vessel and remodel the diseased area. Thereafter, the stent delivery catheter assembly 10 is advanced over the guide wire 23 so that the stent is positioned in the target area. During positioning and throughout the procedure, the partially annealed stent 16 may be visualized through x ray fluoroscopy and/or magnetic resonance angiography.

FIGS. 2 and 3 illustrate cross-sectional views of the catheter assembly 10 at the distal end of the shaft 11 pre-balloon 14 and at the balloon 14, respectively. In FIG. 2, the outer tubular member 19 forms an inflation lumen 21 with the inner tubular member 20, which in turn defines the guide wire lumen 22. In FIG. 3, the stent 16 is shown formed around the balloon 14, which may have two layers 30,31. The balloon defines an annular gap 15 about the inner tubular member 20, which houses the guide wire 23.

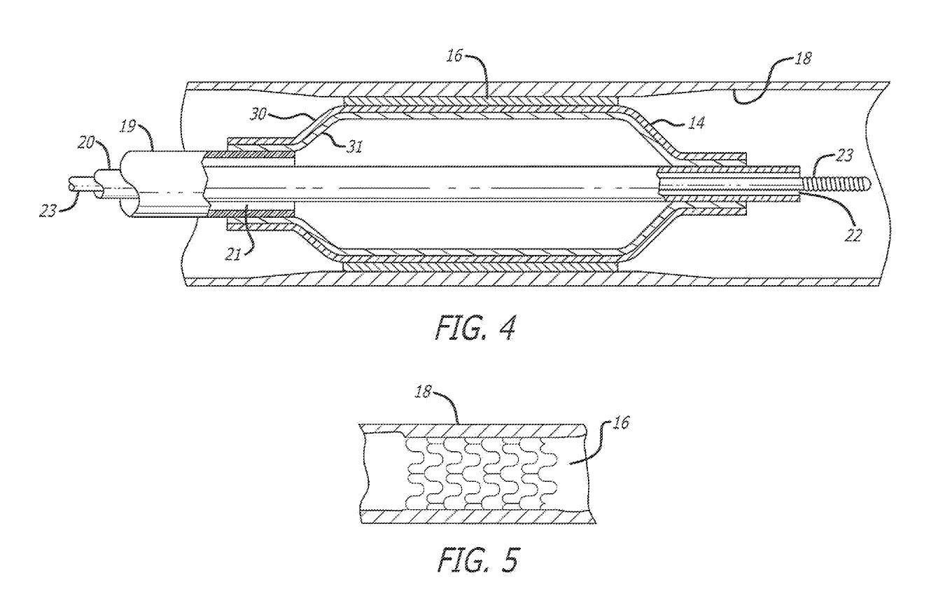

As shown in FIG. 4, the expandable member or balloon 14 is inflated by well known means so that it expands radially outwardly and in turn expands the partially annealed stent 16 radially outwardly until the stent is apposed to the vessel wall 18. The balloon 14 is fully inflated with the stent expanded and pressed against the vessel wall. The balloon is then deflated, and the catheter assembly 10 is withdrawn from the patient's vascular system. The guide wire 23 typically is left in the vessel for post dilatation procedures, if any, and subsequently is withdrawn from the patient's vascular system. As depicted in FIG. 5, the implanted stent 16 remains in the body lumen 18 after the balloon has been deflated and the catheter assembly and guide wire have been withdrawn from the patient.

The stent 16 formed from the partially annealed material serves to hold open the artery wall 18 after the catheter assembly 10 is withdrawn, as illustrated by FIG. 5. Due to the formation of the stent from an elongated tubular member, the undulating components of the stent are relatively flat in transverse cross section, so that when the stent is expanded, it is pressed into the wall of the artery and as a result does not interfere with the blood flow through the artery. The stent is pressed into the wall of the artery and will eventually be covered with endothelial cell growth, which further minimizes blood flow interference. The undulating ring portion of the stent provides good tacking characteristics to prevent stent movement within the artery. Furthermore, the closely spaced cylindrical elements at regular intervals provide uniform support for the wall of the artery, and consequently are well adapted to tack up and hold in place small flaps or dissections in the wall of the artery.

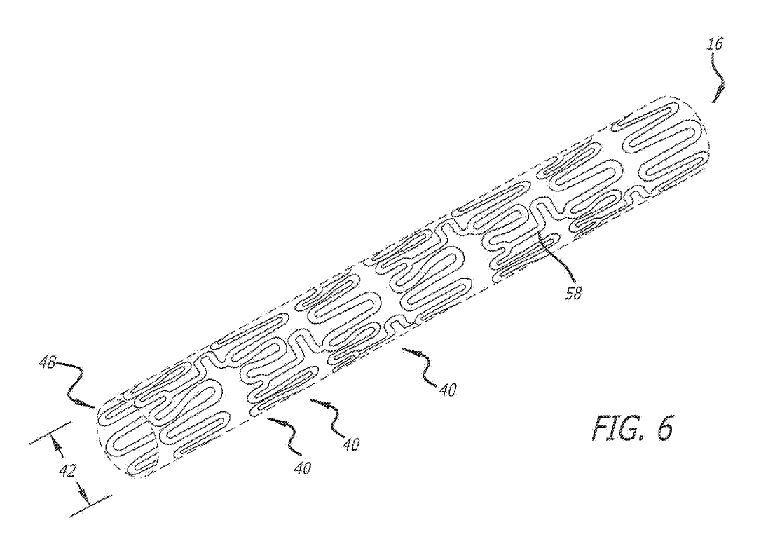

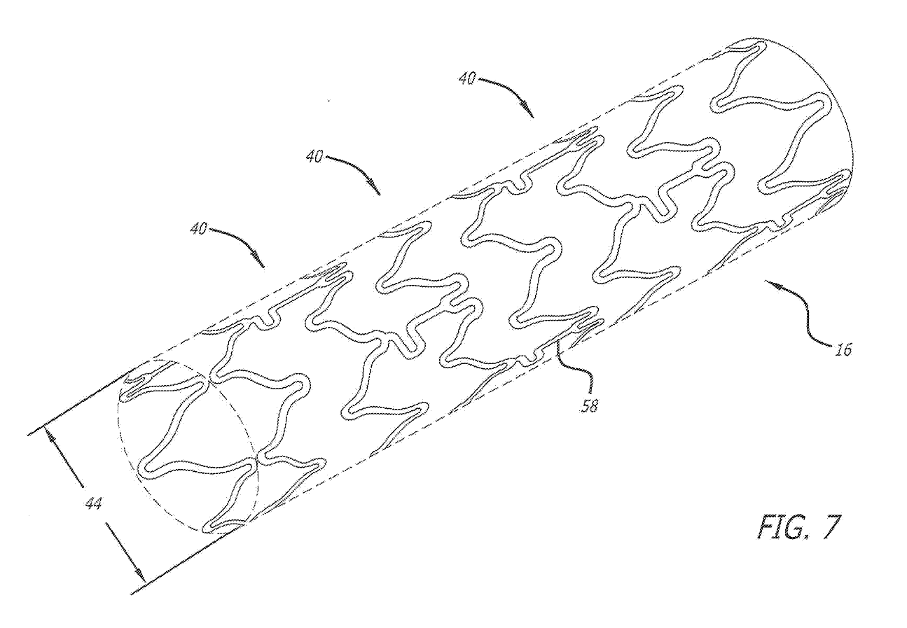

As shown in FIGS. 6-7, the partially annealed stent 16 is made up of a plurality of cylindrical rings 40, which extend circumferentially around the stent. The stent has a delivery diameter 42 (FIG. 6), and an implanted diameter 44 (FIG. 7). When the stent is laser cut from a solid tube, there are no discreet parts, such as the described cylindrical rings. However, it is beneficial for identification and reference to various parts to refer to the cylindrical rings and the following parts of the stent. Each cylindrical ring 40 defines a cylindrical plane 48, which is bound by the cylindrical ring proximal end, the cylindrical ring distal end and the circumferential extent as the cylindrical ring 40 traverses around the cylinder. Each cylindrical ring includes a cylindrical outer wall surface that defines the outer most surface of the partially annealed stent 16, and a cylindrical inner wall surface that defines the innermost surface of the stent. The cylindrical plane 48 follows the cylindrical outer wall surface.

As shown in FIGS. 6 and 7, the stent 16 may be constructed with struts 58 formed from partially annealed material having a variable thickness along the stent length. Each adjacent cylindrical ring 40 may be connected by at least one link 58. The stent 16 may include only straight links, may include only undulating links, or may include links formed of a combination of both undulating sections and straight sections as shown to connect adjacent cylindrical rings 40.

The partially annealed stent 60 of the present invention can be made in many ways. One method of making the stent is to cut a thin walled tube of partially annealed material to remove portions of the tubing in the desired pattern for the stent, leaving relatively untouched the portions of the metallic tubing that are to form the stent. In accordance with the invention, it is preferred to cut the tubing in the desired pattern by means of a machine controlled laser, as is well known in the art. Other methods of forming the stent of the present invention can be used, such as chemical etching; electric discharge machining; laser cutting a flat sheet and rolling it into a cylinder with a longitudinal weld; and the like, all of which are well known in the art at this time. In addition, the stent and/or its struts may be formed from a wire or elongated fiber constructed from a partially annealed material. The cross section of such struts may be round, rectangular or any other suitable shape for constructing a stent.

In the present invention, during the stent manufacturing process the stent material is only partially annealed prior to forming the stent or in which the stent itself is partially annealed after manufacture from work-hardened tubing. This process will somewhat decrease the internal dislocation density caused by drawing, and allow only partial recrystallization. By creating an inhomogeneous grain structure, the partial annealing provides a controllable and optimized balance between strength and ductility of the stent material, resulting in beneficial performance characteristics. This method can be used broadly with any stent material, including stainless steel and cobalt-chromium alloys. And more particularly, testing has shown that the method is particularly useful for some novel refractory metals such as tantalum based alloys. In order to reach an optimal state, the dislocation structure, the recrystallization amount, and the end grain size will be adjusted as necessary to achieve a balance of these properties.

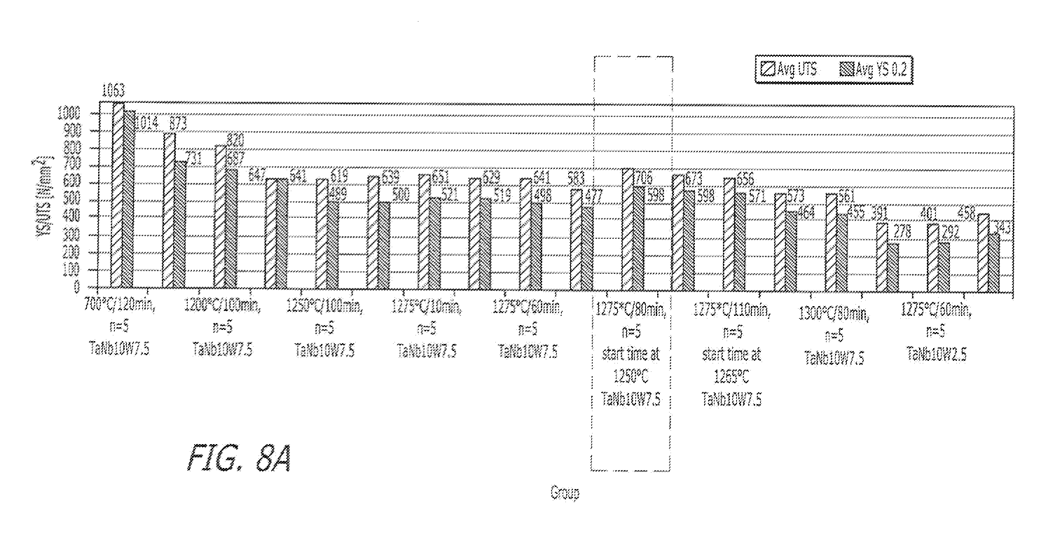

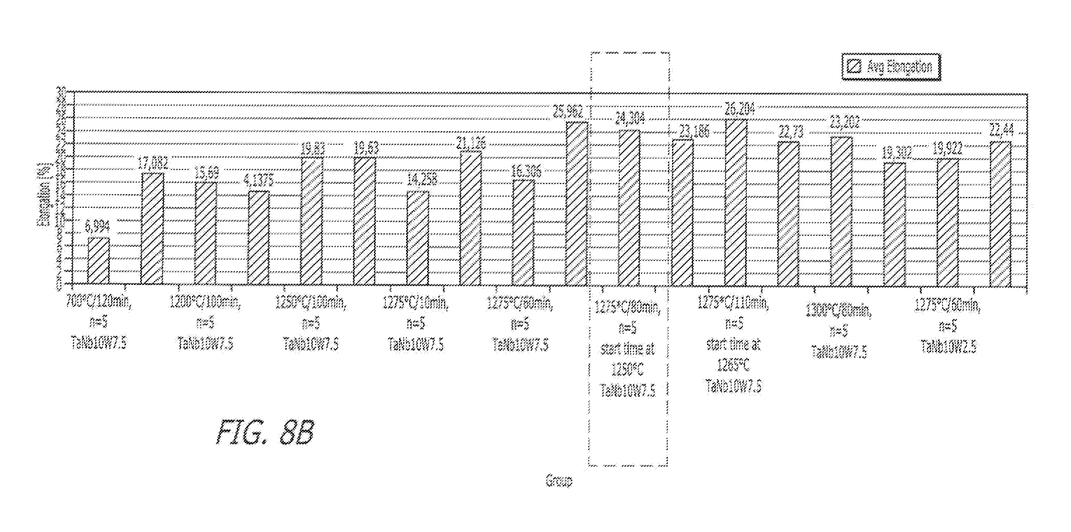

Referring to FIG. 8, testing has been conducted to compare the strength, elongation, micro-hardness, and grain size of a Ta-10Nb-7.5W tantalum alloy (hereafter TaNbW) using different annealing parameters after drawing. The various groups represent wire samples drawn and annealed using the different time and temperature parameters. Each of these groups represent significantly different grain structures, which will result in material behavior that is also significantly different. Returning to FIGS. 8A and 8B, an optimal band of material properties is shown, depending upon the annealing parameters. The optimized band includes material with local maxima (or near maxima) for both strength and elongation. These optimal parameters are dependent on the tubing draw process and material composition. However, the optimal annealing temperatures for the specific TaNbW alloy used for this testing was an annealing process that lasts for 80 minutes at 1275° C. This optimal temperature yielded a material grain size of 12.9 microns (ASTM 9-9.5). This is a much more optimal size for the grains when compared with the size of fully annealed material grains, which were found to be 25.6 microns.

Samples annealed fully using a process that lasted 80 minutes at 1300° C. resulted in material properties that were near a maximum for elongation, but near a minimum for strength. Since stent tubing in the past has been fully annealed, the present invention demonstrates that there is much to be gained by partial annealing.

### EXAMPLE 1

A method is described as illustrative of the present invention. A stent material such as TaNbW is drawn into a tubing form with a residual cold-working of between zero and one hundred percent. The tubing is then annealed to less than full anneal using known annealing processes having time and temperature parameters. The stent tubing is formed into a stent while in the partially annealed state, such as by laser cutting, micromachining, EDM, or photolithography/etching processes. The stent can be fully annealed prior to the final drawing step(s). After the full annealing, there can be at least one or more steps to achieve additional cold work.

While testing was conducted on a TaNb10W7.5 alloy, the partial annealing process of the invention can be applied to any metallic materials used to form stents including stainless steel, cobalt-based alloys, cobalt-chromium alloys, titanium-based alloys, and tantalum alloys.

One example of a tantalum alloy includes a tantalum content of about 77 wt % to about 92 wt %, a niobium content of about 7 wt % to about 13 wt % (e.g., about 7 wt % to about 12 wt %), and a tungsten content of about 1 wt % to about 10 wt %. However, the tantalum alloy may also include other alloying elements, such as one or more grain-refining elements in an amount up to about 5 wt % of the tantalum alloy. For example, the one or more grain-refining elements may include at least one of hafnium, cerium, or rhenium. Tungsten is provided to solid-solution strengthen tantalum, and niobium is provided to improve the ability of tantalum to be drawn. The tantalum alloy is a substantially single-phase, solid-solution alloy having a body-centered cubic crystal structure. However, some secondary phases may be present in small amounts (e.g., inclusions) depending upon the processing employed to fabricate the tantalum alloy.

The composition of the tantalum alloy may be selected from a number of alloy compositions according to various embodiments. In an embodiment, the niobium content is about 8 wt % to about 12 wt % (e.g., about 9 wt % to about 11 wt %), the tungsten content is about 6 wt % to about 9 wt % (e.g., about 6.5 wt % to about 8.5 wt %), and the balance may include tantalum (e.g., the tantalum content being about 80 wt % to about 83 wt %) and, if present, other minor alloying elements and/or impurities. In a more detailed embodiment, the niobium content is about 10 wt %, the tungsten content is about 7.5 wt %, and the balance may include tantalum (e.g., the tantalum content being about 82.5 wt %) and, if present, other minor alloying elements and/or impurities. In another more detailed embodiment, the niobium content is about 10 wt %, the tungsten content is about 2.5 wt %, and the balance may include tantalum (e.g., the tantalum content being about 87.5 wt %) and, if present, other minor alloying elements and/or impurities.

In another embodiment, the niobium content is about 10.5 wt % to about 13 wt %, the tungsten content is about 5.0 wt % to about 6 wt %, and the balance may include tantalum (e.g., the tantalum content being about 80 wt % to about 82 wt %) and, if present, other minor alloying elements and/or impurities. In a more detailed embodiment, the niobium content is about 12.5 wt %, the tungsten content is about 5.8 wt %, and the balance may include tantalum (e.g., the tantalum content being about 81 wt % to about 81.5 wt %) and, if present, other minor alloying elements and/or impurities.

Further embodiments of the process of the present invention may be used for partially annealing materials using other metals and alloys, by varying the annealing temperature and time to achieve the desired degree of partial annealing. Additional example alloys for which the partial annealing manufacturing method of the present invention may be applied include, but are not limited to:

Stainless steels (e.g., 316L stainless steel) may be partially annealed by heating the metal to an annealing temperature ranging between about 800° C. and about 1100° C. and holding the metal at the annealing temperature for a period of time sufficient to achieve the desired degree of partial annealing. L 605 (ASTM F90 and AMS 5759), a Co—Cr—W—Ni alloy also available as STELLITE 25 (Deloro Stellite Company, Inc., South Bend, Ind., U.S.A.) and HAYNES 25 (Haynes International Inc., Kokomo, Ind., U.S.A.), which may be heated to an annealing temperature ranging between about 1120° C. and about 1230° C., and must have rapid cooling (e.g., air) in order to avoid precipitation of undesirable phases.

ELGILOY (ASTM F1058), a Co—Cr—Mo—Ni alloy available from Elgiloy Specialty Metals Division of Elgin, Ill., U.S.A., which may be heated to an annealing temperature ranging from about 1090° C. to about 1150° C.

Platinum iridium (Pt Ir) alloys, which may be heated to an annealing temperature ranging from about 1000° C. to about 1200° C. for alloys having up to ten percent iridium, and ranging from about 1300° C. to about 1500° C. for alloys having greater than ten percent iridium.

Nickel-titanium (Ni Ti) alloys (e.g., nitinol having stoichiometry around 50-50 for shape memory properties), which may be heated to an annealing temperature ranging from about 650° C. to about 950° C., with longer hold times for the lower temperatures

Titanium (Ti) and titanium based alloys, such that pure titanium is heated to an annealing temperature ranging from about 650° C. to about 750° C., with temperatures for titanium alloys depending on the particular alloy.

Instead of working with a semi-annealed tube, it is also within the scope of the present invention to start with a semi- or full-hard tube and control and only partially anneal the tube in the post processing. Alternatively, post-processing steps such as polishing and passivation methods may be used to improve the stent surface finish, as is well known in the art. It may also be necessary to perform a post-processing annealing step. This post-processing annealing step could also be a partially annealing step in accordance with the invention.

While a particular form of the invention has been illustrated and described, it will be apparent to those skilled in the art that various modifications can be made without departing from the spirit and scope of the invention. Accordingly, it is not intended that the invention be limited except by the appended claims.

What is claimed is:

1. A method for manufacturing a stent, comprising:
providing a material for manufacturing a stent;
cold working the material to form tubing;
partially annealing the tubing to less than a full anneal; and
cutting a stent pattern into the partially annealed tubing.

2. The method of claim 2, wherein the material is a stainless steel or cobalt-based alloy.

3. The method of claim 1, wherein the material is an alloy containing tantalum, niobium and tungsten

4. The method of claim 1, wherein the material is a tantalum alloy including:
a tantalum content of about 77 weight % (“wt %”) to about 92 wt %;
a niobium content of about 7 wt % to about 13 wt %; and
a tungsten content of about 1 wt % to about 10 wt %.

5. The method of claim 4, wherein the tantalum content of the tantalum alloy is about 80 wt % to about 83 wt %, wherein the niobium content of the tantalum alloy is about 9 wt % to about 11 wt %, and wherein the tungsten content of the tantalum alloy is about 6.5 wt % to about 8.5 wt %.

6. The method of claim 4, wherein the tantalum content of the tantalum alloy is about 82.5 wt %, wherein the niobium content of the tantalum alloy is about 10 wt %, and wherein the tungsten content of the tantalum alloy is about 7.5 wt %.

7. The method of claim 1, wherein the partial annealing process includes heating the tubing to approximately 1275° C. for 80 minutes.

8. The method of claim 1, wherein the partial annealing process includes heating the tubing to a temperature in the range from 1200° C. to 1300° C. for a time period in the range of 10 minutes to 110 minutes.

9. The method of claim 1, wherein the material is an alloy comprising up to 10 percent by weight of Nb, up to 7.5 percent by weight of W, and a balance of Ta.

10. A method of manufacturing a stent, comprising:
providing a material for manufacturing a stent;
cold working the material to form tubing;
cutting a stent pattern into the tubing; and
partially annealing the tubing with the stent pattern therein.

11. The method of claim 10, wherein the material is a stainless steel or cobalt-based alloy.

12. The method of claim 10, wherein the material is an alloy containing tantalum, niobium and tungsten.

13. The method of claim 12, wherein the material is Ta-10Nb-7.5W by weight.

14. The method of claim 10, wherein the material is an alloy comprising 1 to 10 percent by weight of Nb, 1 to 7.5 percent by weight of W, and a balance of Ta.

15. The method of claim 10, wherein the material is an alloy comprising up to 10 percent by weight of Nb, up to 7.5 percent by weight of W, and a balance of Ta.

16. The method of claim 10, wherein the partial annealing process includes heating the tubing to approximately 1275° C. for 80 minutes.

17. The method of claim 10, wherein the partial annealing process includes heating the tubing to a temperature in the range from 1200° C. to 1300° C. for a time period in the range of 10 minutes to 110 minutes.

18. An arterial stent, comprising:
a series of cylindrical rings joined by connecting struts, the stents formed of a material that is partially annealed.

19. The arterial stent of claim 18, wherein the material is a tantalum alloy.

20. The arterial stent of claim 19, wherein the material is an alloy containing tantalum and niobium.

21. The arterial stent of claim 20, wherein the material is an alloy of tantalum, niobium and tungsten.

22. The arterial stent of claim 21, wherein the material is an alloy comprising up to 10 percent by weight of Nb, up to 7.5 percent by weight of W, and a balance of Ta.
